(19) United States
(12) Patent Application Publication
(10) Pub. No.: US 2021/0301253 A1
(43) Pub. Date: Sep. 30, 2021

(54) METHOD AND APPARATUS FOR SELF-CULTURING AUTOLOGOUS STEM CELLS

(71) Applicant: MEDIKAN CO., LTD., Seoul (KR)

(72) Inventor: Hee-Young LEE, Seoul (KR)

(73) Assignee: MEDIKAN CO., LTD., Seoul (KR)

(21) Appl. No.: 17/262,054

(22) PCT Filed: Aug. 26, 2019
(86) PCT No.: PCT/KR2019/010821

§ 371 Date: Jan. 21, 2021

(30) Foreign Application Priority Data
Sep. 19, 2018 (KR) 10-2018-0111910

(51) Int. Cl. C12N 5/074; B01L 3/00; G16H 80/00

(52) U.S. Cl. CPC C12N 5/0607; C12N 2500/30; G16H 80/00; B01L 3/54

(57) ABSTRACT

Provided is an autologous culture method of autologous stem cells according to an embodiment of the present disclosure, the autologous culture method including: (a) accommodating one's own stem cells collected by a medical institution and a first culture medium in a first culture container from the medical institution to a one's own culture device; (b) controlling the culture device to establish an environment suitable for culture of stem cells; (c) observing the stem cells cultured in the first culture container of the culture device; (d) replacing the first culture medium in the first culture container; (e) separating cultured stem cells from the first culture container; and (f) accommodating separated stem cells in a second culture container with a second culture medium. Accordingly, as an individual cultures one's own stem cells by oneself, costs may be remarkably reduced, a replacement of one's own stem cells with other's stem cells may be fundamentally prevented, and an environment suitable for the culture of one's own stem cells may be provided.

### TECHNICAL FIELD

The present disclosure relates to a stem cell culture method and apparatus, and more particularly, to an autologous culture method and apparatus of autologous stem cells.

### BACKGROUND ART

Recently, the treatment of diseases by collecting human stem cells, culturing the stem cells, and administering the stem cells to the human body is becoming a new treatment method through various evidences.

Conventionally, the entire process of collecting, culturing, administering, and the like of the stem cells has been carried out in medical institutions.

However, since stem cells are cultivated by medical experts in medical institutions, the costs of stem cells cultivation are excessively increased, which makes it difficult to generalize the treatment methods using stem cells.

Furthermore, when a medical institution collects stem cells and uses the stem cells to treat a disease, there may be a problem in that one's stem cells are accidentally replaced with other's stem cells.

Furthermore, stem cells often show a large difference in the growth rate for each individual, it is necessary to establish an environment for each individual for cultivation of stem cells. In medical institutions, it is difficult to precisely match the stem cells culture environment for each individual because independent culture systems are not operated for each individual.

In consideration of the above points, there is a need for persons who want to treat their diseases by culturing and administering their own stem cells to receive safe and optimal stem cells treatment at low cost.

### DESCRIPTION OF EMBODIMENTS

### Technical Problem

Provided is an autologous culture method and apparatus of autologous stem cells according to the present disclosure, which may remarkably reduce costs as an individual cultures one's own stem cells by oneself.

Provided is an autologous culture method and apparatus of autologous stem cells according to the present disclosure, which may fundamentally prevent replacement of one's own stem cells with others stem cells as an individual cultures one's own stem cells by oneself.

Provided is an autologous culture method and apparatus of autologous stem cells according to the present disclosure, which may provide an environment suitable for the culture of one's own stem cells as an individual cultures one's own stem cells by oneself.

The technical objectives to be achieved by the disclosure are not limited to the above-described objectives, and other technical objectives that are not mentioned herein would be clearly understood by a person skilled in the art from the description of the present invention.

### Solution to Problem

According to an aspect of the present disclosure, one who desires to treat a disease by using one's own stem cells performs collection of one's own stem cells by visiting a medical institution to request the collection (medical institution collecting), transferring the collected one's own stem cells outside the medical institution and culturing the same by oneself (autologous culturing), and administering the cultured one's own stem cells by visiting the medical institution to request administering (medical institution administering).

In the process, autologous culturing of stem cells, that is, a simple manipulation such as accommodating stem cells and a culture medium in a culture container, observing a culture process of stem cells, replacing a culture medium, separating stem cells, replacing a culture container, and the like, is performed by an ordinary person, not medical professionals, and if necessary, the medical professionals may remotely observe the culture process of stem cells and give advice a countermeasure.

As an ordinary person performs culturing autologous stem cells by oneself with consultation of medical professionals, costs for culturing may be much reduced, and moreover, erroneously changing one's own stem cells to other's stem cells in a medical institution in a process of curing the stem cells may be fundamentally prevented, and thus stability may be obtained and an optimal culturing environment for a culture process of one's own stem cells may be established by oneself. In particular, determining individual matching of stem cells is impossible in living cells, and even when some samples are used, the costs and time required for genetic testing may serve as an important factor thereby missing treatment timing. In this regard, switching the stem cells in the stem cell culture process may be a very fatal problem so that a need to fundamentally prevent the problem becomes greater.

The failure of culturing stem cells or the generation of contamination of stem cells due to the failure of proper management of one's own stem cells by an ordinary person during a culture process may be easily prevented through remote observation of the culture process and countermeasures by medical professionals in advance.

Accordingly, while the autologous culturing of autologous stem cells may easily prevent the side effects, the effect thereof is very great, and thus the autologous culturing of autologous stem cells may be greatly and actively generalized, much contributing to the treatment of diseases. As a similar example, while autologous insulin injections, autologous hormone injections, and autologous tattoos may easily prevent the side effects, the effect thereof is very great, and thus a general treatment may much contribute to patient treatment, and the like.

According to an embodiment of the present disclosure, an autologous culture method of autologous stem cells includes (a) accommodating one's own stem cells collected by a medical institution and a first culture medium in a first culture container from the medical institution to a one's own culture device, (b) controlling the culture device to establish an environment suitable for culture of stem cells, (c) observing the stem cells cultured in the first culture container of the culture device, (d) replacing the first culture medium in the first culture container, (e) separating cultured stem cells from the first culture container, and (f) accommodating separated stem cells in a second culture container with a second culture medium.

The first culture medium of operation (a) may include a culture medium exclusive of a heterogeneous animal derived material.

The first culture container of operation (a) may include an identification device capable of identifying stem cells.

The identification device may include any one selected from the group consisting of barcodes, RFIDs, special shapes, colors, fingerprints, and photos.

Operation (a) may further include transferring the first culture container containing the stem cells and the first culture medium from the medical institution to the one's own culture device.

The transferring of the first culture container may further include heating the first culture container to maintain the first culture container at a predetermined temperature in the transferring process.

Operation (a) may further include authorizing the stem cells contained in the first culture container.

Operation (b) may include controlling at least any one selected from the group consisting of a pressure, a temperature, and humidity in the culture device.

When at least any one selected from the group consisting of a pressure, a temperature, and humidity in the culture device is out of an allowable range, an alarm signal may be transmitted to a server of a medical institution or a computer or mobile communication terminal device of an autologous culturist, via a network communication network.

In operation (c), the observing of stem cells may be performed by an image capturing apparatus.

An image of stem cells captured by the image capturing apparatus may be transmitted to a server of a medical institution or a computer or mobile communication terminal device of an autologous culturist, via a network communication network.

The replacing of the first culture medium in operation (d) may be automatically performed by supplying a new first culture medium to an inside of the first culture container, without opening the culture device, and simultaneously discharging an existing first culture medium out of the first culture container.

The first culture medium that is discharged may be added with at least one selected from the group consisting of oxygen, water, carbon dioxide, sodium hydroxide, hydrochloric acid, sugar, and protein and then collected back to the first culture container through a collection pipe.

The autologous culture method may further include providing a second culture container accommodating the separated stem cells and the second culture medium to the medical institution.

The autologous culture method may further include checking, by the medical institution that received the second culture container, whether an inside of the second culture container is contaminated or the stem cells in the second culture container is identical to an authorized cell.

The autologous culture method may further include removing an indicator containing a phenol component or removing a heterogeneous animal derived material from the stem cells.

According to another embodiment of the present disclosure, a self-cultivation device of autologous stem cells includes an accommodation portion for accommodating a first culture container containing one's own stem cells collected by a medical institution and a first culture medium in one's own culture device from the medical institution, a control unit for controlling the culture device to establish an environment suitable for the culture of stem cells, an observation portion for observing the stem cells cultured in the first culture container of the culture device, a replacement portion for replacing the first culture medium in the first culture container, a separation portion for separating the cultured stem cells from the first culture container, and a discharge portion for discharging the separated stem cells to be contained in a second culture container with a second culture medium.

### Advantageous Effects of Disclosure

One operation and effect of the autologous culture method and apparatus of autologous stem cells according to the present disclosure is to remarkably reduce costs as an individual cultures one's own stem cells by oneself.

Another operation and effect of the autologous culture method and apparatus of autologous stem cells according to the present disclosure is to fundamentally prevent replacement of one's own stem cells with other's stem cells as an individual cultures one's own stem cells by oneself.

Another operation and effect of the autologous culture method and apparatus of autologous stem cells according to the present disclosure is to provide an environment suitable for the culture of one's own stem cells as an individual cultures one's own stem cells by oneself.

The effects of the present disclosure are not limited to the contents disclosed herein, and other various effects that are not mentioned herein would be clearly understood by a person skilled in the art from the description of the present invention.

### BRIEF DESCRIPTION OF DRAWINGS

FIG. 1 is a flowchart of an autologous culture method of autologous stem cells, according to an embodiment of the present disclosure.

FIG. 2 is a flowchart of a process before an operation of accommodating in a culture device a first culture container containing stem cells and a first culture medium in the flowchart of FIG. 1.

### BEST MODE

Embodiments are provided to further completely explain the disclosure to one of ordinary skill in the art to which the disclosure pertains. However, the disclosure is not limited thereto and it will be understood that various changes in form and details may be made therein without departing from the spirit and scope of the following claims. That is, descriptions on particular structures or functions may be presented merely for explaining embodiments of the disclosure.

### Embodiment 1

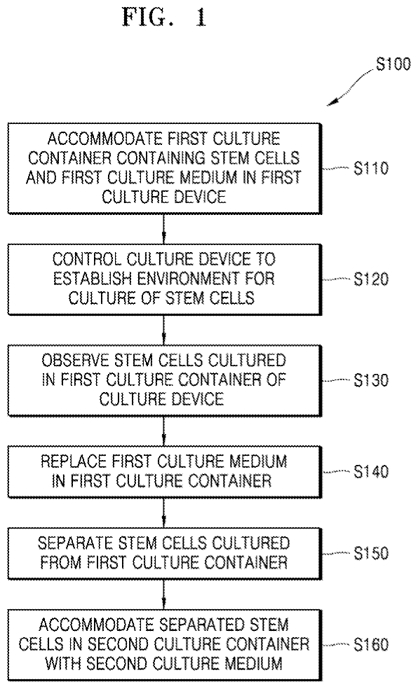

An autologous culture method of autologous stem cells according to an embodiment of the present disclosure (S100) may include, as exemplarily illustrated in FIG. 1, (a) accommodating a first culture container containing one's own stem cells collected by a medical institution and a first culture medium provided by the medical institution in a one's own culture device (S110); (b) controlling the culture device to establish an environment suitable for the culture of stem cells (S120); (c) observing the stem cells cultured in the first culture container of the culture device (S130); (d) replacing the first culture medium in the first culture container (S140); (e) separating the cultured stem cells from the first culture container (S150); and (f) accommodating the separated stem cells in a second culture container with a second culture medium (S160).

First, in operation (a), the first culture container containing the one's own stem cells collected by the medical institution and the first culture medium provided by the medical institution are accommodated in the one's own culture device (S110).

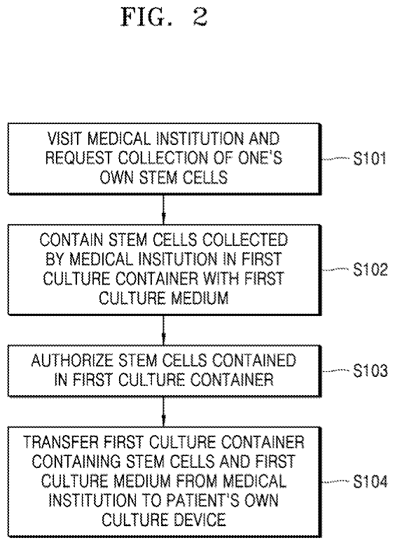

Prior to operation (a), as exemplarily illustrated in FIG. 2, a patient visits a medical institution such as a hospital and requests collection of one's own stem cells (S101).

Upon the request of the patient, in the medical institution, a predetermined amount of predetermined human tissue, for example, 10 cc of fat tissue, 5 cc of marrow, 200 cc of blood, 2 cm² of skin, and the like, is sucked from the patient by using a stem cells collection device, and materials other than stem cells are filtered from the sucked human tissue to collect a predetermined stem cell. The above collected stem cells are contained in the first culture container with the first culture medium (S102).

The first culture container may include an identification device capable of identifying stem cells, for example, the owner of the contained stem cell, and the like. The identification device may include various devices capable of identifying stem cells. For example, the identification device may any one selected from the group consisting of barcodes, RFIDs, special shapes, colors, fingerprints, and photos.

The first culture container may include a member such as a sealed vacuum bottle to prevent the contained stem cells from being contaminated by foreign materials included in the air, and the like. For example, the first culture container may include a sealed bag including polyethylene (PE) or polyethylene terephthalate (PET).

The first culture container may include a light shield material to prevent the stem cells from light.

The first culture medium for culturing the collected stem cells is provided by a medical institution. The first culture medium may include various materials capable of culturing stem cells. However, the first culture medium may be a culture medium exclusive of the heterogeneous animal derived material to prevent a bad influence to the stem cells due to the heterogeneous animal derived material.

The medical institution may further perform authorization of the stem cells contained in the first culture container (S103). In other words, the medical institution may request authorization of the stem cells contained in the first culture container that satisfy a predetermined reference from an external organization and obtain an authorization. However, the authorization is not limited thereto, and the authorization may be obtained by the medical institution, not by the external organization. As such, due to the authorization, the reason for the contamination of stem cells, and the like in the subsequent operations may be found from other places, not in the medical institution.

As such, in the medical institution, the first culture container containing the stem cells and the first culture medium is transferred from the medical institution to the patient's own culture device (S104). The transfer is made by a transfer device that transfers the first culture container containing the stem cells and the first culture medium. A process of transferring the first culture container containing the stem cells and the first culture medium from the medical institution to the patient's own culture device transfer may be performed while maintaining the stem cells and the first culture medium contained in the first culture container at a predetermined temperature. To maintain the stem cells and the first culture medium contained in the first culture container at the predetermined temperature, the transfer device may include a temperature control device that maintains the first culture container at the predetermined temperature. When the first culture container is maintained at the predetermined temperature by the temperature control device, the stem cells and the first culture medium contained in the first culture container may be transferred while being maintained at the predetermined temperature. For example, when an outside temperature increases higher than the predetermined temperature so that the temperature of the first culture container increases higher than the predetermined temperature, the temperature control device cools the first culture container so that the temperature of the first culture container maintains the predetermined temperature. When the outside temperature decreases lower than the predetermined temperature so that the temperature of the first culture container decreases lower than the predetermined temperature, the temperature control device heats the first culture container so that the temperature of the first culture container maintains the predetermined temperature.

The first culture container containing the one's own stem cells collected by the medical institution and the first culture medium provided by the medical institution is transferred by the transfer device to the patient's own culture device and is accommodated in the culture device (S110).

In operation (b), the culture device is controlled to establish an environment suitable for the culture of stem cells (S120).

The control of the culture device means controlling the culture device to establish an environment suitable for the culture of stem cells contained in the first culture container with the first culture medium. An environment condition suitable for the culture of stem cells to be controlled by the culture device may include various conditions. For example, the control of the culture device may include controlling at least any one selected from the group consisting of pressure, temperature, and humidity in the culture device.

When the environment condition in the culture device, for example, at least any one selected from the group consisting of pressure, temperature, and humidity is out of an allowable range, an autologous culturist may control the culture device such that pressure, temperature and humidity in the culture device belong to the allowable range. In this state, when at least any one selected from the group consisting of the pressure, temperature, and humidity in the culture device is out of the allowable range, an alarm signal may be transmitted to a sever of a medical institution connected via a wired/wireless network communication network or a computer or mobile communication terminal device of an autologous culturist. Accordingly, the medical institution server transmits an alarm signal or an alarm signal and countermeasures to a computer or mobile communication terminal device of an autologous culturist connected via the wired/wireless network communication network, and thus the autologous culturist quickly controls the culture device to allow the pressure, temperature, and humidity in the culture device to be within the allowable range, thereby preventing damage to the stem cells cultured in the culture device and inducing continuous culture of the stem cells.

In operation (c), the stem cells cultured in the first culture container of the culture device is observed (S130).

The observing of the stem cells cultured in the first culture container of the culture device may be performed in various methods. For example, the observing of stem cells may be performed by an image capturing apparatus. The image capturing apparatus captures an image of stem cells by magnifying the same so that it may be checked with the naked eye how much the stem cells are cultured. Furthermore, the image capturing apparatus may capture images of stem cells in a culture process only at specific time points per hour or continuously in time. An autologous culturist may identify a culture state of stem cells according to a result of the observing of stem cells and may replace the first culture medium or control the culture device according to the culture state of stem cells, thereby taking an action such as controlling environment conditions for the culture of stem cells, and the like.

An image of stem cells captured by the image capturing apparatus may be transmitted to a server of a medical institution or a computer or mobile communication terminal device of an autologous culturist via a wired/wireless network communication network. Accordingly, the medical institution server transmits images of stem cells, or images of stem cells and countermeasures, to a computer or mobile communication terminal device of an autologous culturist connected via a wired/wireless network communication network, and thus the autologous culturist may control the culture device to replace the first culture medium or change environment conditions for culture of stem cells such as a pressure, a temperature, and humidity in the culture device, thereby promoting the culture of stem cells in the culture device. In this state, the countermeasures may include pieces of advice to the autologous culturist to take an action based on a result of observation of stem cells by medical professionals such as doctors, nurses, researchers, and the like of medical institutions. When adult stem cells are mutated or contaminated, the entire cells die or are changed into the form of colony, which may be easily identified through images of stem cells.

In operation (d), the first culture medium in the first culture container is replaced (S140). Accordingly, the stem cells contained in the first culture container may be smoothly cultured by the first culture medium that is replaced.

The replacement of the first culture medium may be performed by various methods. For example, the first culture medium may be replaced by opening the culture device, supplying a new first culture medium to the inside of the first culture container, and sealing the culture device. However, the present disclosure is not limited thereto, and the first culture medium may be replaced without opening the culture device by supplying a new first culture medium to the inside of the first culture container and simultaneously discharging the existing first culture medium out of the first culture container. The replacement of a culture medium may be automatically performed. To this end, the culture device may include, in the first culture container, a discharge pipe for discharging the existing culture medium and simultaneously a culture medium storage container for separately storing a culture medium, the culture medium storage container being connected to the first culture container through an inlet pipe. Accordingly, by discharging the first culture medium from the first culture container through the discharge pipe to the outside and simultaneously introducing a new culture medium from the culture medium storage container to the first culture container through the inlet pipe, a containment possibility may be reduced and the culture medium may be automatically replaced.

Furthermore, the first culture medium discharged out of the first culture container may be added with at least any one selected from the group consisting of oxygen, water, carbon dioxide, sodium hydroxide, hydrochloric acid, sugar, and protein and may be collected back to the first culture container through a collection pipe. Accordingly, usage efficiency of the culture medium may be much improved.

In operation (e), the cultured stem cells may be separated from the first culture container (S150).

The separation of cultured stem cells may mean that the cultured stem cells that are attached to the first culture container are separated from the first culture container.

In operation (f), the separated stem cells are accommodated in a second culture container with a second culture medium (S160).

In addition, the autologous culture method of autologous stem cells (S100) according to the present disclosure may further include providing the medical institution with a second culture container in which the separated stem cells and a second culture medium are accommodated.

It may be further included that the medical institution that received the second culture container accommodating the separated stem cells and the second culture medium checks whether the inside of the second culture container is contaminated or whether stem cells in the second culture container is identical to the authorized stem cells. Furthermore, it may be further included that the medical institution that received the second culture container accommodating the separated stem cells and the second culture medium removes an indicator containing a phenol component or removing heterogeneous animal derived materials from the stem cells.

### Embodiment 2

The present embodiment relates to a self-cultivation device of autologous stem cells.

The self-cultivation device of autologous stem cells may include: an accommodation portion for accommodating a first culture container containing one's own stem cells collected by a medical institution and a first culture medium in one's own culture device from the medical institution; a control unit for controlling the culture device to establish an environment suitable for the culture of stem cells; an observation portion for observing the stem cells cultured in the first culture container of the culture device; a replacement portion for replacing the first culture medium in the first culture container; a separation portion for separating the cultured stem cells from the first culture container; and a discharge portion for discharging the separated stem cells to be contained in a second culture container with a second culture medium.

The first culture container accommodated in the accommodation portion may include an identification device for identifying stem cells. The identification device may include any one selected from the group consisting of barcodes, RFIDs, special shapes, colors, fingerprints, and photos.

The control unit may include controlling at least any one selected from the group consisting of a pressure, a temperature, and humidity in the culture device. When the at least any one selected from the group consisting of a pressure, a temperature, and humidity in the culture device is out of an allowable range, the control unit may transmit an alarm signal to a server of a medical institution or a computer or mobile communication terminal device of an autologous culturist via a communication network.

The observing of stem cells by the observation portion may be performed by using an image capturing apparatus. An image of stem cells captured by the image capturing apparatus may be transmitted through the network communication network to a server of a medical institution or a computer or mobile communication terminal device of an autologous culturist.

The replacement of the first culture medium by the replacement portion may be automatically performed by supplying a new first culture medium to the inside of the first culture container, without opening the culture device, and simultaneously discharging the existing first culture medium out of the first culture container. The discharged first culture medium may be added with at least one selected from the group consisting of oxygen, water, carbon dioxide, sodium hydroxide, hydrochloric acid, sugar, and protein, and then collected back to the first culture container through a collection pipe.

### INDUSTRIAL APPLICABILITY

The present disclosure may be used for a stem cell culture method and apparatus.

1. An autologous culture method of autologous stem cells, the autologous culture method comprising:
(a) accommodating one's own stem cells collected by a medical institution and a first culture medium in a first culture container from the medical institution to a one's own culture device;
(b) controlling the culture device to establish an environment suitable for culture of stem cells;
(c) observing the stem cells cultured in the first culture container of the culture device;
(d) replacing the first culture medium in the first culture container;
(e) separating cultured stem cells from the first culture container; and
(f) accommodating separated stem cells in a second culture container with a second culture medium.

2. The autologous culture method of claim 1, wherein
the first culture medium of operation (a) comprises a culture medium exclusive of a heterogeneous animal derived material.

3. The autologous culture method of claim 1, wherein
the first culture container of operation (a) comprises an identification device capable of identifying stem cells.

4. The autologous culture method of claim 3, wherein
the identification device comprises any one selected from the group consisting of barcodes, RFIDs, special shapes, colors, fingerprints, and photos.

5. The autologous culture method of claim 1, wherein
operation (a) further comprises transferring the first culture container containing the stem cells and the first culture medium from the medical institution to the one's own culture device.

6. The autologous culture method of claim 5, wherein
the transferring of the first culture container further comprises heating the first culture container to maintain the first culture container at a predetermined temperature in the transferring process.

7. The autologous culture method of claim 1, wherein
operation (a) further comprises authorizing the stem cells contained in the first culture container.

8. The autologous culture method of claim 1, wherein
operation (b) comprises controlling at least any one selected from the group consisting of a pressure, a temperature, and humidity in the culture device.

9. The autologous culture method of claim 8, wherein,
when at least any one selected from the group consisting of a pressure, a temperature, and humidity in the culture device is out of an allowable range, an alarm signal is transmitted to a server of a medical institution or a computer or mobile communication terminal device of an autologous culturist, via a network communication network.

10. The autologous culture method of claim 1, wherein,
in operation (c), the observing of stem cells is performed by an image capturing apparatus.

11. The autologous culture method of claim 10, wherein
an image of stem cells captured by the image capturing apparatus is transmitted to a server of a medical institution or a computer or mobile communication terminal device of an autologous culturist, via a network communication network.

12. The autologous culture method of claim 1, wherein
the replacing of the first culture medium in operation (d) is automatically performed by supplying a new first culture medium to an inside of the first culture container, without opening the culture device, and simultaneously discharging an existing first culture medium out of the first culture container.

13. The autologous culture method of claim 12, wherein
the first culture medium that is discharged is added with at least one selected from the group consisting of oxygen, water, carbon dioxide, sodium hydroxide, hydrochloric acid, sugar, and protein and then collected back to the first culture container through a collection pipe.

14. The autologous culture method of claim 1, further comprising
providing a second culture container accommodating the separated stem cells and the second culture medium to the medical institution.

15. The autologous culture method of claim 14, further comprising
checking, by the medical institution that received the second culture container, whether an inside of the second culture container is contaminated or the stem cells in the second culture container is identical to an authorized cell.

16. The autologous culture method of claim 1, further comprising
removing an indicator containing a phenol component or removing a heterogeneous animal derived material from the stem cells.
